(19) United States
(12) Patent Application Publication
(10) Pub. No.: US 2008/0257361 A1
(43) Pub. Date: Oct. 23, 2008

(54) MALE CONDOM WITH TENSION BAND

(75) Inventor: Lawrence S. Hakim, Weston, FL (US)

Correspondence Address: DENNIS T. GRIGGS, 17950 PRESTON ROAD, SUITE 1000, DALLAS, TX, 75252, US

(73) Assignee: CDS International Holdings, Inc., Delray Beach, FL (US)

(21) Appl. No.: 11/737,659

(22) Filed: Apr. 19, 2007

(51) Int. Cl. A61F 6/04; A61H 1/02

(52) U.S. Cl. 128/844; 600/38

(57) ABSTRACT

A single-use, disposable condom utilizes a fully customizable and adjustable tension band to apply clamping pressure around and about the base of an erect penis in order to facilitate maximal corporal veno-occlusive function, thereby enabling a stronger erection, as well as for custom tightening at the base of the penis without completely restricting flow. To prevent re-use of the condom and tension band, the tension band includes manually operable fasteners that can be permanently attached and bonded to the base of the condom sheath itself after the tension band has been adjusted. The bond between the tension band and the sheath is sufficiently strong enough to resist separation forces that exceed the rupture strength of the condom sheath and the breaking force of the tension band. In one embodiment, the tension band includes one or more lobes that selectively focus clamping pressure onto the dorsal vein and thereby restrict the drainage of venous blood from engorged penile tissue.

### BACKGROUND OF THE INVENTION

1. Field of the Invention

The present invention relates generally to contraceptive devices, and in particular to a male condom featuring an improved tension band that provides ease of application and removal, increased sensation, superior protection against communicable disease and unplanned pregnancy caused by condom leakage around the base, and the ability to prolong erection.

2. Description of the Prior Art

The function of a male condom is to form a barrier around the penis, the male organ of sexual intercourse, to prevent direct contact of body parts and fluids that might transmit disease or cause pregnancy. There are inherent limitations in the design of the conventional condom that result in reduced condom acceptance and usage in the population. For example, the contact barrier and snugness of the fit limit tactile sensorial abilities. The decrease in feeling reduces the user's pleasurable sensations, and therefore inhibits the desire to use a condom.

Sexually-transmitted diseases affect millions of people. Because of the threat of AIDS, and other sexually transmitted diseases, condoms are enjoying unprecedented popularity. The male condom is marketed as one of the few over-the-counter, nonprescription contraceptives to be used by the male during sexual intercourse. The FDA continues to promote the use of condoms as the most effective method of protection against certain sexually transmitted diseases. A condom is a device, usually made of latex, or more recently polyurethane, that is used by the male partner during sexual intercourse. It is worn on a male's penis, for the purposes of preventing pregnancy and preventing the transmission of sexually transmitted diseases (STDs) such as gonorrhea, syphilis and HIV.

According to U.S. government agency reports, for example, as published by the CDC (Centers for Disease Control), in the USA alone, 10 percent of all reported AIDS cases occur among people over the age of 50, with a quarter of these over 60. The dominant risk factors are the same as for other age groups, including heterosexual sex, unprotected sex and multiple sexual partners.

Studies indicate that older persons tend to view condom usage primarily as a means of contraception, not to prevent STDs. Therefore, women who are no longer concerned about unplanned pregnancy may not insist on regular condom usage. A recent US study estimated that only one out of six sexually active persons over the age of 50 is likely to use a condom. The reasons for this are attributable, at least in part, to limitations in the design of the conventional male condom.

Published reports indicate that over 50% of men between the ages of 40-70 years suffer from some degree of erectile dysfunction (ED), or the inability to attain and/or maintain an erection that is sufficient for satisfactory intercourse (MMAS, Feldman et al, 1994). In addition, despite the development of effective therapeutic options for some men with ED, for example, sildenafil citrate sold under the brand Viagra™ by Pfizer Corporation, the majority of men with erectile dysfunction remain untreated. The fact remains that with the current design of male condoms, a firm erection is required prior to placement.

Moreover, in order for the male condom to perform its function, including primarily as a barrier against pregnancy and STDs, an erection must be maintained during use. Conventional condom usage is clearly predicated on the male having normal erectile function. Otherwise, the male condom cannot be put on easily or properly, and can loosen or pull off prematurely during use, resulting in pregnancy or the spread of disease. As a result of this inherent design limitation, many men are unable to, or refuse to use conventional male condoms during sexual intercourse.

In spite of FDA warnings and the seriousness of the risks involved, a significant number of sexually active males and females of all ages decline to use condoms or use them only occasionally, for a variety of reasons. Condoms may feel unnatural and uncomfortable, or may interfere with spontaneity. They may inhibit sexual arousal. They are difficult to use by men who are experiencing erectile dysfunction.

The average length of an erect male penis is six inches. Standard condoms extend 7.5 inches (excluding reservoir tip). This length is sufficient to accommodate the needs of 90% of the male population. In fact, 95% of all men have erect penises less than 8.5 inches long. The circumference of an erect penis also varies within the population. The majority of males have a penis circumference measuring between 87.5 mm (3½ inches) and 150 mm (6 inches).

Most condoms sold in the United States closely conform to the same basic shape. These condoms are designed for a snug fit around the penis from the tip, or just below the tip, down to the base. A commonly available condom size sold in the United States is approximately 190 mm (7.5 inches) long (including reservoir tip), by 104 mm or 108 mm (approx 4 inches) in circumference. Another condom, of conventional design sold in Japan is 165 mm (approx. 6½ inches) by 87.5 mm (approx. 3½ inches) in circumference.

A common feature of conventional condoms is that they provide a snug fit. Many of these condoms have a reservoir tip which helps guard against leakage around the base by forming a repository for ejaculate.

Conventional condoms, because they are form-fitting, are difficult to put on and are awkward to remove. Most condoms are applied by the rolling-on method. The condom should be applied to the erect penis before any sexual contact. The condom is fitted about the head of the penis and completely unrolled to the base of the penis, ideally using the fingers with dexterity. However, many times this is a difficult process and the fingers may cause skin irritation. The process is more difficult, if not impossible, if erectile function is diminished. This process can cause the condom to catch and pinch skin and pull pubic hair during application (and removal), possibly causing condom rupture. Because of the requirement for tight fit, condoms often develop a vacuum during the removal process, adding to the difficulty of removal.

The utility period of a condom usually begins after the penis is erect and fully expanded, and ends just after ejaculation as the erection subsides. The utility time period does not extend beyond ejaculation; following ejaculation, the penis should be withdrawn promptly while still erect. Manufacturers warn that the condom is likely to slip off as the penis becomes flaccid following ejaculation, allowing seminal fluid to spill out, defeating protection against STDs or unplanned pregnancy. Thus, prompt withdrawal and removal of the condom after use is uniformly recommended by manufacturers of conventional condom devices in order to avoid leakage and fluid contact.

A problem that is commonly encountered during use of conventional condoms available today is that for many, the ability to sustain an erection while wearing or even putting on a condom can be difficult, and quickly turns a special moment of passion into one of despair. There is nothing more embarrassing for a man than to lose his erection during sexual activity. In addition, an estimated 40 million men in the USA, both young and old, have experienced the frustration of erectile dysfunction or “ED,” which makes conventional condom usage almost impossible.

This is especially critical in patients with any significant degree of corporal veno-occlusive dysfunction, which causes diminished capacity or inability to store blood adequately in the penis during erection. These are contributing reasons that many men, especially those with any degree of erectile dysfunction, often refuse to or choose not to wear conventional condoms during intercourse. Unfortunately, this may expose both men and their partners to the significant risks of sexually transmitted diseases, including HIV and AIDS, as well as the risk of unplanned pregnancies, thereby contributing to a high rate of abortion in the world today.

In condom design, sensorial limitations are mitigated somewhat by using thinner materials of construction. However, although a thinner material improves heat conduction and enhances tactile sensation, these thinner materials tend to be less reliable, and may increase the likelihood of condom failure by rupture.

### BRIEF SUMMARY OF THE INVENTION

The innovative single-use, disposable condom design of the present invention utilizes a fully customizable and adjustable tension band which is supplied separately or incorporated into the condom itself, secured about the proximal end of the condom for engagement around the base of the penis. The tension band is designed to apply clamping pressure around and about the base of the penis in order to facilitate maximal corporal veno-occlusive function, thereby enabling a stronger erection, as well as for custom tightening at the base of the penis without completely restricting flow.

According to a preferred embodiment of the invention, to prevent re-use of the condom and tension band, the tension band includes a fastener portion that can be permanently attached and bonded to the base of the condom sheath itself after the tension band has been adjusted to provide an effective clamping action. Once the tension band has been manually adjusted to a comfortable fit, it is permanently attached and bonded on its opposite end to the base of the condom. The bond between the free end and the sheath or between the free end and the linking portion is sufficiently strong enough to resist separation forces that exceed the rupture strength of the condom sheath and the breaking force of the tension band.

This permanent attachment ensures that the condom will be ruptured and the tension band will be broken if an attempt is made to separate the band from the condom, thereby preventing re-use of the condom and the tension band. This is important because condom use and tension band use should be limited to a one-time use in order to prevent spread of infection and disease.

According to another embodiment of the invention, an adjustable tension band includes one or more lobes that apply local pressure onto the dorsal vein and thereby restrict the drainage of venous blood from engorged penile tissue.

Preferably, the adjustable tension band is secured around the base of the condom by mating strips of hook and loop fastener members. After an effective clamping force is produced, the union of the fastener members is made permanent by adhesive deposits that are carried on or incorporated into the adjustable tension band, permanently attaching the tension band onto the condom for restricting venous drainage as a means to enhance erectile function during use with a condom. In this embodiment, the adhesive bond is sufficiently strong enough to resist separation forces that exceed the rupture strength of the condom sheath and the breaking force of the tension band.

According to another aspect of the invention, a permanent attachment of the tension band is made by a fast-setting binary epoxy contact adhesive which bonds so tightly that it is stronger than the condom sheath material and the tension band material. The binary components of the contact adhesive are applied to the free end of the tension band and to the condom sheath during manufacture, and are shielded against contact by peel-off release strips until ready for use.

The closure of the male condom according to the invention is accomplished by a band of a elastic or inelastic fastening material which is wrapped around the base of the condom and then is fastened onto itself by deposits of fast-setting binary epoxy contact adhesive which bonds so tightly that it is stronger than the tension band and the condom sheath material.

The seals produced by condoms fitted with tension bands according to the invention, which protect against insemination and transmission of disease, are provided by the tension band clamping action, which can cinch down tightly to minimize the likelihood of leakage at the base. Enhanced and prolonged erections are enabled by this clamping action, which contracts tightly enough to restrict the drainage of venous blood from the penis, both before and after ejaculation, thereby maintaining erection by dorsal veno-occlusion and extending the time of use.

### BRIEF DESCRIPTION OF THE DRAWINGS

FIG. 1 is a perspective view of a male condom equipped with a tension band of the present invention shown in the rolled-up configuration, prior to placement on a penis;

FIG. 2 is a perspective view of the male condom of FIG. 1, in the unrolled, extended configuration, having a tension band equipped with Velcro™ fasteners shown in the relaxed, unfastened condition prior to tensioning about the condom sheath;

FIG. 3 is a perspective view of the male condom of FIG. 2, shown fitted and fully deployed about an erect male penis, as it may appear during intercourse;

FIG. 4 is a perspective view of a tension band constructed according to an alternative embodiment of my invention;

FIG. 5 is a sectional view of the tension band taken along the line 5-5 of FIG. 4;

FIG. 6 is a side elevation view of the tension band of FIG. 4, fitted onto a condom about the base of an erect penis which is shown in arterial relief;

FIG. 7 is a detailed cross-section view through an erect penis, taken along the line 7-7 of FIG. 6;

FIG. 8 is a detailed cross-section view through the tension band of FIG. 4 clamped about an erect penis, taken along the line 8-8 of FIG. 6;

FIG. 9 is a top plan view of a tension band constructed according to an alternative embodiment of my invention;

FIG. 10 is a longitudinal cross-section view through the tension band of FIG. 9, taken along the line 10-10 of FIG. 9; and

FIG. 11 is a detailed cross-section view showing the tension band of FIG. 9 fitted in tension and clamped engagement about a condom and made permanent by an adhesive deposit.

### DETAILED DESCRIPTION OF THE PREFERRED EMBODIMENT

A disposable male condom 10 according to the present invention is fitted with a tension band 12 that prolongs penile erection during intercourse, without interfering with application and removal of the condom, while accommodating the effective use of the condom for its primary purpose as a prophylactic contraceptive and disease barrier.

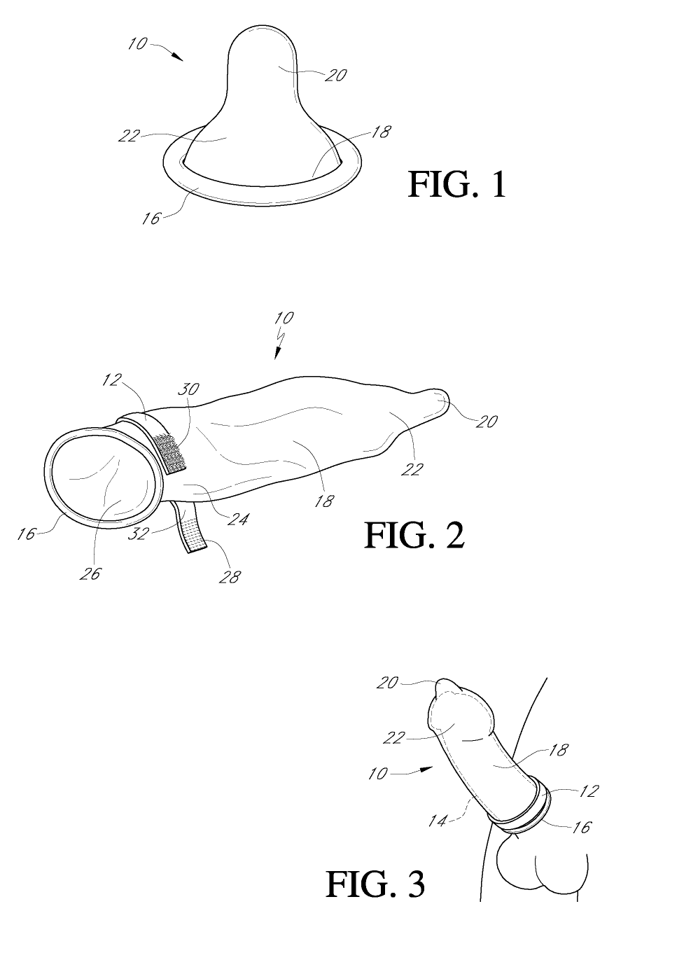

In FIG. 1 there is shown the male condom 10 of the present invention just removed from packaging and before application about an erect penis 14. The male condom 10 has two principal components: a condom base ring 16, a tubular sheath 18 and a closure tip 20 formed on its distal end portion 22 that provides a reservoir for collecting ejaculate.

A proximal base end portion 24 of the male condom sheath 18 borders the condom base ring 16 and forms an opening 26 for receiving an erect penis 14. The tension band 12 is fitted about the proximal end base portion 24 of the male condom 10, closely adjacent the base ring 16. The tension closure band 12 is attached to the sheath 14 at the base 22 with its length tightly circumscribing and clamping the proximal end base portion about the erect penis 14.

According to the preferred embodiment of the invention, the tension band 12 is constructed of a soft, comfortable and durable material such as latex rubber or polyurethane, preferably fabricated as part of the condom itself as shown in FIG. 2, with Velcro™ male and female fasteners 28, 30 integrally formed in the tension band 12 or in the underlying proximal sheath portion 24, or coupled thereto by linking members 32, 34 that are integrally formed with the tension band. Optionally, the tension band can be constructed of an inelastic material.

The tension band 12 is approximately 2-3 cm (approx. 1 inch) in width and extends approximately 10 cm (approx. 4 inches) in length. The linking portions 32, 34 together with overlapping potions of the Velcro™ male and female fasteners 28, 30 at the base 24 of the condom allow custom fitting about a range of penis sizes.

After an erection develops, the man or his partner places the condom onto the head of the penis 14 and rolls it down along the phallus, in the same way that a conventional condom is fitted. Referring again to FIG. 2, when the tension band is in a relaxed condition, the condom base 24 has a full opening 26 for receiving the head of the penis. After the condom 10 has been rolled down over the erect shaft of the penis 14, and excess air has been removed, the proximal base portion 22 of the penis is clamped by the tension band 12, thereby reducing venous drainage flow at the base of the penis.

As shown in FIG. 2 and FIG. 3, after the condom 10 has been rolled on, the tension band 12 is wrapped tightly and secured around the base 24 of the erect penis 14. The male end 28 of a Velcro™ fastener strip (which is located at the tip of the tension band) overlaps and attaches to the female-end 30 of a Velcro™ fastener strip, thus providing a custom fit.

Once in place, the tension band 12 can then be easily adjusted snugly and comfortably using a Velcro™ (or comparable) hook and loop fastening device, allowing for a custom fit for every man, every time, and enabling the man to better sustain his erection by facilitating maximal corporal veno-occlusive function. Once sexual intercourse is completed, the tension band 12 is pulled away and separated from the condom, and the condom 10 and the tension band 12 are discarded.

The disposable, safe-release condom 10 and tension band can be used with any commercially available personal lubricants designed for condom usage. All of the condom and tension band surfaces exposed during use are completely smooth and comfortable.

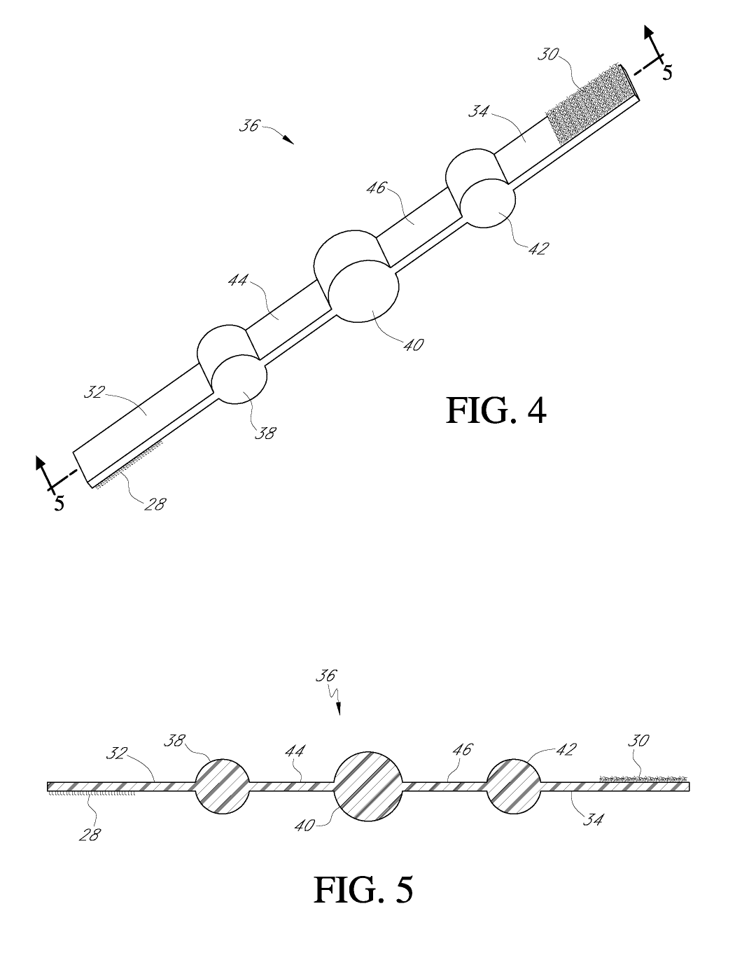

According to an alternative embodiment of my invention, shown in FIG. 4 and FIG. 5, an adjustable tension band 36 includes one or more lobes, preferably three lobes 38, 40 and 42 that apply local pressure onto the dorsal vein of the penis 14 and thereby restrict the release and drainage of venous blood from engorged tissue. Preferably, the lobes 38, 40, 42, referred to as the left ventral lobe, the dorsal lobe and the right ventral lobe, are integrally formed in an elongated elastic band formed by linking portions 44, 46 which connect the lobes and fasteners together.

The lobes 38, 40, 42 when pulled inwardly by constriction of the tension band, apply clamping pressure on the spongy erectile tissue chamber regions 48, 50, 52 that are inflated and expanded with blood during an erection. Preferably, the lobes have a curved profile in cross section, as shown in FIG. 4, and have an enlarged radial thickness and are made of a relative high durometer polyurethane material that is capable of compressing the ventral and dorsal tissues 48, 50 and 52, as illustrated in FIG. 8.

The radial projecting lobe portions are used advantageously as indexing means by the man to properly position the dorsal lobe 40 over the dorsal veins 58, 60. The left and right ventral lobes 38, 40 will be properly positioned adjacent the left ventral spongy erectile tissue region 48 and right ventral spongy erectile tissue region 52 when the dorsal lobe is properly positioned over the dorsal veins.

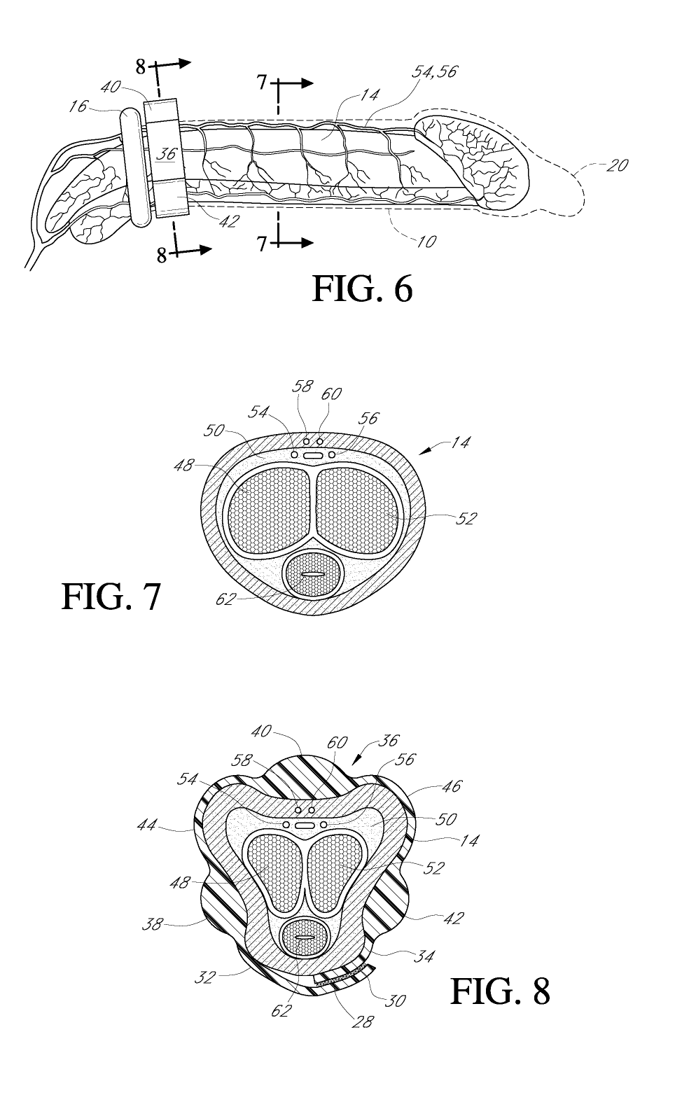

Referring now to FIG. 6, FIG. 7 and FIG. 8, the primary physiological mechanism that brings about erection is the autonomic dilation of the two dorsal arteries 54, 56 that supply blood to the penis, which allows more blood to fill the three spongy erectile tissue chambers 48, 50, 52, causing the shaft of the penis to lengthen and stiffen. The engorged erectile tissue presses against and constricts the veins 58, 60 that carry blood away from the penis. More blood enters than leaves the penis until an equilibrium condition is reached where an equal volume of blood flows into the dilated arteries 54, 56 and out of the constricted veins 58, 60, thereby achieving a constant erectile size at equilibrium.

As the tension band 36 is tightened against the base of the penis, the ventral lobes 48, 52 and the dorsal lobe 50 compress the spongy erectile tissue chamber regions 48, 50, 52 inwardly, thus producing a resultant clamping force that is focused on the veins 58, 60 that carry blood away from the penis, preventing outflow and thus sustaining the erection. Because of the triangular positioning of the lobes, the clamping force is directed generally away from the urethra 62. This is a beneficial consequence, because it is generally desirable that the urethra should remain relatively uncompressed to permit the free discharge flow of ejaculate.

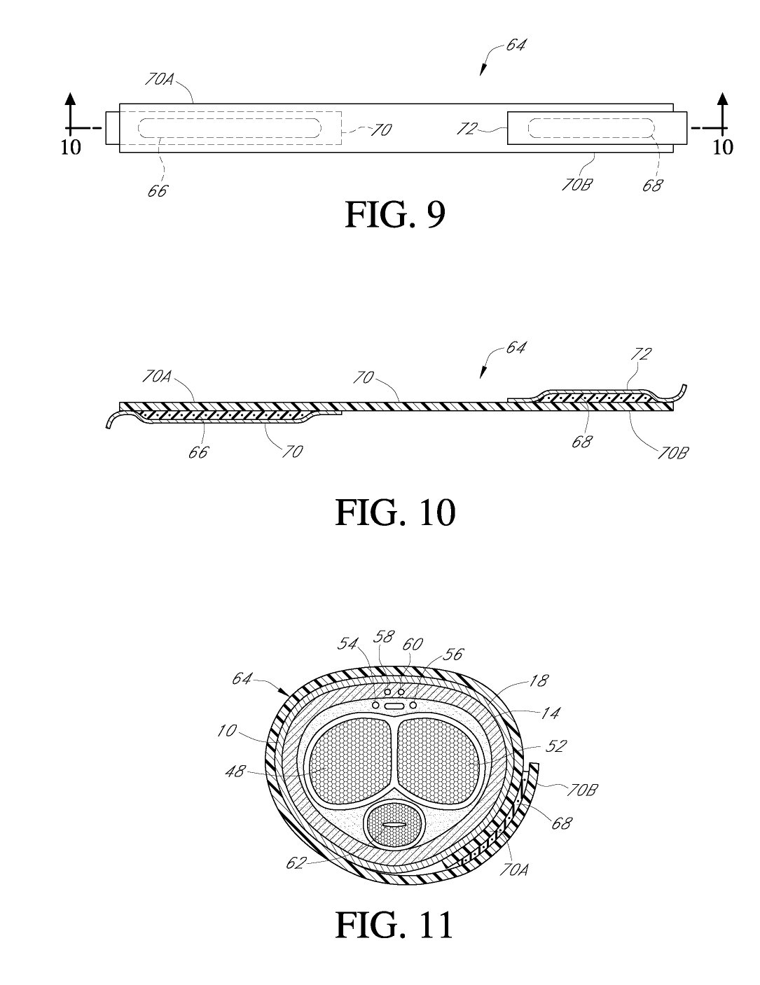

A tension band 64 constructed according to an alternative embodiment of my invention is shown in FIG. 9, FIG. 10 and FIG. 11. The tension band 64 includes fastener portions 66, 68 that are applied manually to provide effective and comfortable clamping action. To prevent re-use of the condom 10 and tension band, the tension band 64 is permanently set and bonded to the base 24 of the condom sheath 18 itself after the tension band has been adjusted.

Once the tension band 64 has been manually adjusted to a comfortable and effective fit, it is permanently attached and bonded on its opposite ends to the proximal base 24 of the condom 10. The bond between the end portions 70, 71 and the sheath 18 and between the overlapping end portions is sufficiently strong enough to resist separation forces that exceed the rupture strength of the condom sheath and the breaking force of the tension band.

This permanent attachment ensures that the condom sheath 18 will be ruptured and the tension band 64 will be broken if an attempt is made to separate the band from the condom 10, thereby preventing re-use of the condom and the tension band. This is important because condom use and tension band use should be limited to a one-time use in order to prevent spread of infection and disease.

In particular, adhesive deposits can be employed, for example as shown in FIG. 9 and FIG. 10. Preferably, the adhesive deposit is medical grade Cyanoacrylate. Cyanoacrylate is the generic name for methyl-2-cyanoacrylate, which is used in medical applications, in products such as Dermabond™ medical glue and Traumaseal™ medical glue. Cyanoacrylate adhesives are known as instant adhesives and are ideally suited to provide permanent bonding for this tension band 64 embodiment.

Cyanoacrylate is an acrylic resin which rapidly polymerizes in the presence of moisture (specifically hydroxide ions), forming long, strong chains, joining the bonded surfaces together. Because the presence of moisture causes the glue to set, exposure to moisture in the air can cause a tube or bottle of glue to become unusable over time. To prevent an opened container of glue from setting before use, it must be stored in an airtight container. Cyanoacrylate sets fast, and an accelerator such as toluidine trigger setting in two or three seconds.

The safe-release condom 10, equipped with a tension band 12, or 36, or 64 as described herein, will help all men, with or without erectile dysfunction, to better maintain their erections. This will allow the man to prolong the act of intimacy for both partners, while preventing the man from losing his erection too quickly.

Since its use is not regulated, the condom of the present invention can also be used as an adjunct to medical therapy, such as Viagra™, Levitra™, Cialis™ or Caverject™, to safely facilitate the male partner's erection, allowing it to remain even stronger and better maintained than with drugs alone.

For the condom sheath 18, the material of construction must be able to withstand push and pull forces of intercourse. A high quality latex, such as, Sheerlon® brand latex, which combines strength and maximum protection against breaking or tearing, and thinness for comfort and superior sensation is preferred. All disclosed embodiments of condoms and tension bands according to my invention can be made of polyurethane, thermoplastic elastomer (TPE), natural rubber latex, or other suitable natural or synthetic materials.

A fast acting, strong contact adhesive material is preferred, so that the condom sheath will be ruptured or torn in response to release of the tension strap as the male condom 10 removed, and the tension band will also be broken or pulled apart in response to the destructive release action during removal of the condom.

Since the adhesive material forms a permanent bond, protective guard release strips 70, 72 cover the adhesive deposits until the erect penis 12 is inserted within the male condom 10 and ready to be clamped by the tension band 64. After the penis is fully inserted onto the condom, the guard strip 60 is peeled off and the exposed adhesive deposit is applied against the condom sheath. After allowing a few seconds to allow the adhesive to bond with the sheath 18, the tension band 64 is pulled tightly around the base of the penis until a comfortable and effective clamping pressure is developed. Then, the remaining guard strip 72 is peeled off, and the exposed adhesive deposit 68 is contacted against the top surface of the tension band end portion 70A.

The application procedure for the male condom 10 is easy and fast, causing little interruption. To don the male condom 10, the wearer slips the bottom 22 over the erect penis 12, and holding the external band end 32 of the tension band 12, pulls it tightly around the base of the erect penis 12 to overlap internal band end 30. This seals closure 20 and bottom 22 comfortably and securely about the base of the penis 12 in the constricted condition.

When thus sealed, if sufficient tension was applied when the band ends 30 and 32 were overlapped, the venous flow of blood from the erect penis 12 will be restricted, which can cause maintenance of erection, possibly beyond ejaculation.

To doff male condom 10, the man pulls on the exterior band end 70B thereby breaking the seal at the proximal base portion of the condom, rupturing the sheath 18 and breaking the tension band 64. At this point, the male condom 10 can be easily pulled off.

While in use, the tension band 12 is in its constricted state creating a tight viscous seal around the base of the erect penis 12, as shown in FIG. 3, FIG. 6 and FIG. 11. It is this seal which prevents transmission and commingling of fluids between the partners.

The tension band 64 includes an elongated band section 70 preferably made of durable material, which may be elastically resilient, or stretchable, or non-stretchable inelastic material, and which may generally have the same physical properties as the band material of the embodiments shown in FIGS. 1-8. The tension band 64 is fastened to the outer surface of the condom sheath 18, preferably only in the region closely adjacent the proximal end 24 of the condom 10.

One feature of the tension band closure embodiments of the present invention is that they can constrict the condom base over a range of various arbitrarily small diameters, thereby facilitating the establishment of an effective seal, and being capable of effectively restricting venous blood flow, even in the case of unusually small diameter penis sizes, as well as those having a large or above average diameter. In all embodiments of the invention, the condom can have some tapering in the vicinity of the tip 20.

In some embodiments described herein, the tension band is either integrally formed with the condom or is permanently anchored thereto by an adhesive bond, wherein the adhesive bond has a strength greater than the rupture strength of the condom sheath and greater than the breaking strength of the tension band. In all embodiments the tension band may or may not be elastically resilient, or stretchable. The lobed embodiment may be used successfully with mechanical fasteners only, and can also be used successfully with or without an adhesive deposit.

I claim:

1. A disposable condom comprising:
a sheath including a closed end portion, a receiving base portion and a tubular sidewall member extending between the closed end portion and the receiving base portion;
a tension band including an anchor portion, a movable end portion and a flexible linking portion extending between the anchor portion and the movable end portion;
manually operable, mutually engagable fastener means coupled to the anchor portion and to the movable end portion for securing the flexible linking portion in wrapped tension engagement at least partially around the sheath, the fastener means when engaged forming a permanent connection between the anchor portion and the moveable end portion that is sufficiently strong enough to resist separation forces that exceed the rupture strength of the condom and the breaking force of the tension band.

2. A disposable condom as set forth in claim 1, wherein the fastener means comprises:
a selected one of a male fastener or a female fastener coupled to the condom sheath;
a selected one of a male fastener or a female fastener coupled to the tension band;
a deposit of adhesive disposed on one of the selected fasteners; and
a manually removable release strip covering the adhesive deposit.

3. A disposable condom as set forth in claim 1, wherein the fastener means comprises:
a patch of flexible hooks; and
a patch of flexible loops.

4. A disposable condom as set forth in claim 1, wherein the fastener means comprise male and female coupling members capable of interlocking engagement, and
a selected one of the male or female coupling members is attached to the condom sheath;
a selected one of the male or female coupling members is attached to the tension band;
an adhesive deposit is disposed on a selected one of the male or female coupling members, or on a convoluted turn of the tension band, for forming a permanent union between the male or female coupling members or the tension band when the tension band is stretched in tension about the condom; and
a manually removable release strip is disposed in covering engagement over the adhesive deposit.

5. A disposable condom as set forth in claim 1, the fastener means comprising:
a rapidly curing adhesive that is operative to produce a bonded union between the condom sheath and the tension band that is sufficiently strong enough to resist separation forces that exceed the rupture strength of the condom sheath and the breaking force of the tension band.

6. A disposable condom as set forth in claim 1, wherein the fastener means comprises:
a first adhesive deposit disposed on the anchor portion;
a second adhesive deposit disposed on the movable end portion;
a first manually removable release strip covering the first adhesive deposit; and
a second manually removable release strip covering the second adhesive deposit.

7. A disposable condom as set forth in claim 1, wherein the fastener means comprises:
a first patch of a selected one of hook or loop fasteners;
a second patch of a selected one of hook or loop fasteners;
an adhesive deposit disposed on a selected one of the fastener patches; and
a manually removable release strip covering the adhesive deposit.

8. A disposable condom as set forth in claim 7, the adhesive deposit comprising:
a rapidly curing adhesive that is operative to produce a bonded union between the condom sheath and the tension band that is sufficiently strong enough to resist separation forces that exceed the rupture strength of the condom sheath and the breaking force of the tension band.

9. A disposable condom as set forth in claim 1, wherein the fastener means comprises:
a male fastener member coupled to a selected one of the condom sheath or the tension band for receiving and engaging a female coupling member;
a female fastener member attached to a selected one of the tension strap or the condom sheath;
an adhesive deposit disposed on a selected one of the male fastener or the female fastener for forming a permanent union between the tension band and the condom sheath when the fasteners are disposed in interlocking engagement; and
a manually removable release strip covering the adhesive deposit.

10. A disposable condom comprising, in combination:
a sheath including a closed end portion, a receiving base portion and a tubular sidewall member extending between the closed end portion and the receiving base portion;
a tension band including an elongated body portion and one or more lobes projecting from the body portion for engaging against the receiving base portion of the condom; and
manually operable fasteners adapted for attachment to the condom sheath and to the tension band for securing the tension band in wrapped tension engagement at least partially around the receiving base portion of the condom sheath.

11. A disposable condom as set forth in claim 10, wherein the tension band comprises a plurality of lobes disposed in an operative engaging configuration for applying local pressure against erectile tissue of an erect human penis.

12. A disposable condom as set forth in claim 10, wherein the tension band comprises three lobes symmetrically disposed in an operative engaging configuration for applying local pressure against erectile tissue in the dorsal region and against erectile tissue in the left and right ventral regions of a human penis.

13. A disposable condom comprising, in combination:
a sheath including a closed end portion, a receiving base portion and a tubular sidewall member extending between the closed end portion and the receiving base portion;
a tension band including an anchor portion, a movable end portion and a flexible linking portion extending between the anchor portion and the movable end portion; and
manually operable adhesive fastener means coupled to the anchor portion and to the movable end portion for securing the flexible linking portion in tension engagement at least partially around the sheath, the manually operable adhesive fastener means including:
a first reactive component deposit of a fast-setting binary epoxy adhesive;
a second reactive component deposit of a fast-setting binary epoxy adhesive;
a first manually removable release strip covering the first deposit; and
a second manually removable release strip covering the second deposit.

14. A method for securing a disposable condom that has been fitted about an erect penis comprising the steps:
providing a tension band having an anchor portion, a movable end portion, and a linking portion connecting the movable end portion to the anchor portion;
securing the anchor portion of the tension band onto the base of the condom;
engaging the linking portion of the tension band in tension around the base of the condom; and
forming an adhesive bond between the moveable end portion and the condom or between the moveable end portion and the anchor portion that is sufficiently strong enough to resist separation forces that exceed the rupture strength of the condom and the breaking force of the tension band.

15. A method for sustaining an erection comprising the steps:
providing a tension band that includes one or more lobes;
securing the tension band in wrapped tension engagement around the base of an erect penis; and
applying local pressure through one or more the lobes against the dorsal vein of the erect penis.

16. A method for sustaining an erection as set forth in claim 14, further comprising the steps:
fitting a condom about the erect penis;
attaching an anchor end of the tension band onto the receiving base portion of the condom; and
forming an adhesive bond between the moveable end portion of the tension band and the condom or between the moveable end portion and the anchor portion that is sufficiently strong enough to resist separation forces that exceed the rupture strength of the condom and the breaking force of the tension band.
